(19) United States
(12) Patent Application Publication
(10) Pub. No.: US 2006/0165789 A1
(43) Pub. Date: Jul. 27, 2006

(54) Lercanidipine modified release compositions

(75) Inventors: Wattanaporn Abramowitz, West Windsor, NJ (US); Ram P. Kapil, Princeton Junction, NJ (US); Todd A. Riccobene, Basking Ridge, NJ (US); Mahendra G. Dedhiya, Pomona, NY (US); Yan Yang, Roslyn Heights, NY (US); Anil Chhettry, Holtsville, NY (US)

Correspondence Address: DARBY & DARBY P.C., P. O. BOX 5257, NEW YORK, NY, 10150-5257, US

(73) Assignee: Forest Laboratories, Inc., New York, NY (US)

(21) Appl. No.: 11/223,493

(22) Filed: Sep. 9, 2005

Related U.S. Application Data
(60) Provisional application No. 60/609,224, filed on Sep. 9, 2004.

(51) Int. Cl. A61K 31/445; A61K 9/22

(52) U.S. Cl. 424/468; 514/317

(57) ABSTRACT

Pursuant to the present invention, it has been found that a modified release composition containing the low permeability and poor solubility drug, lercanidipine, may be prepared which provides for therapeutically effective plasma concentrations of lercanidipine for a period of about 20 to about 25 hours. The modified release composition of the present invention provides modified release of lercanidipine independent of pH and therefore provides release of lercanidipine even upon exposure to the low pH use environments, such as gastric fluid.

### CROSS-REFERENCE TO RELATED APPLICATIONS

The present application claims the benefit of priority under 35 U.S.C. § 119(e) of Provisional Application Ser. No. 60/609,224, filed Sep. 9, 2004, which is hereby incorporated herein by reference in its entirety.

### FIELD OF THE INVENTION

The present invention relates to modified release bead compositions that achieve long term release of lercanidipine. The modified release compositions of the present invention deliver lercanidipine with a sustained therapeutic effect compared to presently commercially available lercanidipine containing products. The present invention further provides for unit dosage forms comprising modified release beads of the present invention or a mixture of modified and immediate release beads.

### BACKGROUND OF THE INVENTION

Modified release dosage forms provide a means for once a day dosing thereby improving patient compliance and ensuring effective and safe therapy with minimal side effects. Compared to immediate release dosage forms, modified release dosage forms can be used to prolong pharmacologic action after administration, and to reduce variability in the plasma concentration of a drug throughout the dosage interval, thereby eliminating or reducing sharp peaks. In light of the advantages of modified release dosage forms, it has been the objective of many skilled in the art to develop such dosage forms.

The majority of modified release dosage forms comprise a core either coated with or containing a drug. The core is then coated with a release modifying polymer within which the drug may be dispersed. The release modifying polymer disintegrates gradually, releasing the drug over time. Thus, the outer-most layer of the composition effectively slows down and thereby regulates the diffusion of the drug across the coating layer when the composition is exposed to an aqueous environment, i.e., the gastrointestinal tract. The net rate of diffusion of the drug is mainly dependent on the ability of the gastric fluid to penetrate the coating layer or matrix and on the solubility of the drug itself.

Because the rate of drug diffusion from a modified release dosage form is dependent in part on the solubility of the drug itself, the development of modified release dosage forms for slightly or poorly soluble and low permeable drugs (lercanidipine is such a drug) has proven to be more difficult. Therefore, there remains a need in the art for modified release compositions of low solubility drugs and in particular modified release dosage forms containing the poorly soluble drug lercanidipine, which ensure prolonged therapeutic plasma concentrations and reduce or eliminate peaks in plasma concentration.

Lercanidipine (methyl 1,1,N-trimethyl-N-(3,3-diphenylpropyl)-2-aminoethyl 1,4-dihydro-2,6-dimethyl-4-(3-nitrophenyl)pyridine-3,5-dicarboxylate) is a highly lipophilic dihydropyridine calcium antagonist with a long duration of action and high vascular selectivity. The molecular formula of the hydrochloride salt of lercanidipine is set forth in below.

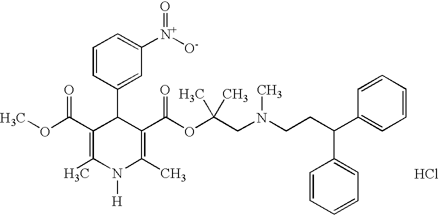

The hydrochloride salt of lercanidipine is commercially available from Recordati S.p.A. (Milan, Italy). Methods of making both lercanidipine free base and its hydrochloride salt have been described previously along with methods of resolving lercanidipine into individual enantiomers in U.S. Pat. Nos. 4,705,797; 5,767,136; 4,968,832; 5,912,351; and 5,696,139, all of which are incorporated herein by reference. Lercanidipine is a dihydropyridine calcium antagonist. As other calcium channel antagonists, it lowers blood pressure by relaxing arteriolar smooth muscle, which decreases peripheral vascular resistance. Lercanidipine produces no negative cardiac inotropism and, occasionally only, mild reflex tachycardia generally of short duration. It has a high affinity for and competitively antagonizes the dihyropyridine subunit of the L-type calcium channel. Lercanidipine has been approved for the treatment of hypertension and has been marketed in several European countries under the trademark Zanidip® since 1996.

Lercanidipine alone or in combination with additional active agents has been shown to be effective in once and twice daily administration. Lercanidipine has been studied in the dosage ranging form 2 to 80 mg. Lercanidipine is normally administered at doses of about 10 mg to about 20 mg once or twice daily in immediate release tablet form. Lercanidipine is used for treating Stage I and Stage II hypertension and is also expected to be useful in alleviating angina pectoris. It has also been beneficial in elderly patients with isolated systolic hypertension. The recommended starting oral dose of lercanidipine is given by mouth 10 mg once daily and is increased, if necessary, after at least 2 weeks to 20 mg daily. Upon oral administration, lercanidipine is absorbed and peak plasma level occurs 1-3 hours following dosage.

Lercanidipine and its salts, such as the hydrochloride salt, are practically insoluble in water, displaying an aqueous solubility of about 5 μg/ml. The solubility of lercanidipine is marginally greater in acidic mediums, however, even at pH 5 it is less than 20 μg/ml. Lercanidipine also shows poor experimental permeability (i.e., poor permeability, Pₐₚₚ of 0.5×10⁻⁷ cm/s, in a Caco2 cell apparatus and low bioavailability) and is classified as a low permeable drug, as defined by the FDA. Additionally, when administered to patients, lercanidipine displays extensive presystemic first pass elimination as a result of its being a substrate for cytochrome P450 3A4 isoenzyme. The combination of poor water solubility, low permeability and considerable first pass metabolism results in low and highly variable bioavailability.

In order to improve the bioavailability of lercanidipine, food can be co-administered with each dosage. The administration of food along with lercanidipine has been shown to increase the absorption of lercanidipine significantly and therefore enhance its efficacy, a phenomenon known as “food effect.” Studies have shown that simultaneous intake of food (especially food having a high fat content) increases the amount of lercanidipine absorbed between three and four times compared to administration without food. The same studies have shown that lercanidipine administered in the absence of food is not entirely absorbed which results in low and variable bioavailability. The dependence of effective dosing and absorption of lercanidipine upon co-administration of food is inherently undesirable and can result in fluctuations in effectiveness, inter-patient variability, and in poor patient acceptance and/or compliance.

Accordingly, in order to facilitate the effective administration of lercanidipine to patients, there is a need in the art for an oral dosage form which results in absorption and ensures greater bioavailability of lercanidipine. Particularly, there is a need for an oral dosage form such that lercanidipine may be administered in the absence of food. More particularly, there is a need for a modified release pharmaceutical composition that provides modified release of lercanidipine.

### SUMMARY OF THE INVENTION

Pursuant to the present invention, it has been found that a modified release composition containing the low permeability and poor solubility drug, lercanidipine, may be prepared which provides for therapeutically effective plasma concentrations of lercanidipine for a period of about 20 to about 25 hours. The modified release composition of the present invention provides modified release of lercanidipine independent of pH and therefore provides release of lercanidipine even upon exposure to the low pH use environments, such as gastric fluid.

Additionally, the modified release composition of the present invention reduces the peak plasma concentration of lercanidipine, while providing long term plasma concentrations at, or above, the therapeutic plasma concentration. This in turn permits increased daily dosing, increasing therapeutic effect with limited or no increase in side-effects.

The present invention provides a modified release bead composition comprising an immediate release core and a first layer comprising at least one release modifying acrylic polymer, wherein the beads have a average radius from about 10 mesh, 2 mm to about 140 mesh, 0.1 mm.

In one embodiment of the present invention the modified release bead compositions comprise (i) an immediate release core comprising (a) an inert core, (b) a first layer comprising a solubility and permeability enhancing surfactant, a binder and lercanidipine, and (c) optionally a second layer comprising a film coating, and (ii) an outer layer comprising (a) at least one release modifying acrylic polymer, and (iii) optionally a film coating.

In yet another embodiment of the present invention, the modified release bead composition releases at least about 80% of the lercanidipine content in vitro, within about 6 hours. The preferred means of determining dissolution of lercanidipine is the USP Basket Method 1,100 RPM in 900 ml aqueous buffer (0.01 N HCl at 37° C.). In another embodiment of the present invention, the modified release bead composition releases at least about 80% of the lercanidipine content in vitro, within about 12 hours. One skilled in art will appreciate that the release rates can be adjusted from 4 to 24 hours.

In yet another embodiment, the modified release beads of the present invention may be combined with immediate release beads to form a unit dosage form, wherein the unit dosage form provides both immediate and modified release of lercanidipine upon administration to a patient, thereby providing for a rapid increase in lercanidipine plasma concentrations following administration and sustained therapeutic plasma concentration levels for a period of about 20 to about 25 hours following administration to a patient. In an alternative embodiment, the modified release beads of the present invention may be combined with immediate release beads and or pH dependent pulsatile beads to form a unit dosage form, wherein the unit dosage form provides both immediate, pulsatile and modified release of lercanidipine upon administration to a patient, thereby providing for a rapid increase in lercanidipine plasma concentrations following administration and sustained therapeutic plasma concentration levels for a period of about 20 to about 25 hours following administration to a patient.

In another embodiment, the present invention provides a unit dosage form comprising immediate and modified release beads wherein upon administration of the dosage form to a patient the peak plasma concentration of lercanidipine is from about 8 ng/ml to about 14 ng/ml and the time to peak concentration is from about 2 to 12 hours following administration of the modified bead composition.

In yet another embodiment the present invention provides a unit dosage form comprising immediate and modified release beads wherein upon administration of the dosage form to a patient the plasma concentration of lercanidipine remains at therapeutic levels, e.g., greater than about 0.1 to about 0.4 mg/mL for a period of about 20 to about 25 hours following administration.

In still another embodiment the present invention provides a method of treating a patient suffering from hypertension by administering the modified release composition disclosed herein, and wherein administration of the composition disclosed herein results in long term plasma concentration of lercanidipine above therapeutic levels.

These and other aspects of the present invention will be apparent to those of ordinary skill in the art in the light of the present description, claims and figures.

### DESCRIPTION OF THE DRAWINGS

FIG. 1 depicts the comparative dissolution profiles of three batches of lercanidipine immediate release pharmaceutical beads having an outer layer comprising Eudragit® RL 30D and Opadry™, the curve represented by -♦- is the dissolution profile of the reference batch, the curve represented by -▴- is the dissolution profile of batch 2 according to the present invention and the curve represented by -▪- is the dissolution profile of batch 3 according to the present invention.

FIG. 2 depicts the comparative dissolution profiles of three batches of lercanidipine modified release pharmaceutical beads having an outer layer comprising a combination of Eudragit® RL 30D and Eudragit® RS 30D, the curve represented by -♦- is the dissolution profile of the reference batch, the curve represented by -▴- is the dissolution profile of batch 2 and the curve represented by -▪- is the dissolution profile of batch 3.

FIG. 3 depicts the comparative dissolution profiles of four formulations of lercanidipine modified release pharmaceutical beads having an outer layer comprising Eudragit® RL 30D and increasing amounts of Opadry™ (Type III modified release beads) the curve represented by -▴- is the dissolution profile of Formulation II containing 15% Opadry™ (by weight of Eudragit®-RL), the curve represented by -●- is the dissolution profile of Formulation III containing 10% Opadry™ (by weight of Eudragit® RL), the curve represented by -▪- is the dissolution profile of Formulation IV containing 5% Opadry™ (by weight of Eudragit® RL), and the curve represented by -♦- is the dissolution profile of Formulation V containing 0% Opadry™.

FIG. 4 depicts the comparative dissolution profiles of six formulations of lercanidipine modified release pharmaceutical beads having an outer layer comprising varying ratios of Eudragit® RL 30D to Eudragit® RS 30D (Type IV modified release beads) the curve represented by -▴- is the dissolution profile of Formulation II containing a ratio of RL:RS of 100:0, the curve represented by -▪- is the dissolution profile of the Formulation III containing a ratio of RL:RS of 90:10, the curve represented by -▴- is the dissolution profile of Formulation IV containing ratio of RL:RS of 80:20, the curve represented by -▴- is the dissolution profile of Formulation V containing a ratio of RL:RS of 70:30, the curve represented by -x- is the dissolution profile of Formulation VI containing a ratio of RL:RS of 60:40, and the curve represented by -●- is the dissolution profile of Formulation VII containing a ratio of RL:RS of 50:50.

FIG. 5 depicts the comparative dissolution profile of a modified release bead formulation and a unit dosage form of the present invention, the curve represented by -▪- is the dissolution profile of the Prototype III capsule, prepared as described in Example 6, and the curve represented by -♦- is the dissolution profile of a modified release bead of Type III formulation VI having an outer layer comprising Eudragit® RL 30D and Opadry™.

FIG. 6 depicts the comparative dissolution profile of a modified release bead formulation and a unit dosage form of the present invention, the curve represented by -▪- is the dissolution profile of the Prototype IV capsule, prepared as described in Example 6, and the curve represented by -♦- is the dissolution profile of a modified release formulation of Type IV formulation VII having an outer an outer layer comprising a combination of Eudragit® RL 30D and Eudragit® RS 30D.

### DETAILED DESCRIPTION OF THE INVENTION

As used herein, the following terms are defined as follows:

The term “about” means within 10% of a given value, preferably within 5%, and more preferably within 1% of a given value. Alternatively, the term “about” means that a value can fall within a scientifically acceptable error range for that type of value, which will depend on how qualitative a measurement can be given the available tools.

The phrase “dissolution profile” as used herein, refers to the dissolution of an agent over time. The dissolution can be measured as relative amount agent dissolved over time, the amount of agent dissolved, or the concentration of the agent.

The term “lercanidipine” means the free base composition methyl 1,1,N-trimethyl-N-(3,3-diphenylpropyl)-2-aminoethyl 1,4-dihydro-2,6-dimethyl-4-(3-nitrophenyl)pyridine-3,5-dicarboxylate, as well as any pharmaceutically acceptable salt, e.g., a salt with an inorganic or organic acid such as, HCl, HBr, H₂SO₄, maleic acid, fumaric acid, tartaric acid and citric acid. Preferred pharmaceutically acceptable salts of lercanidipine include, but are not limited to, hydrochloride, besylate and napadisylate salts. Additionally, lercanidipine may be present in crystalline and/or amorphous forms. Preferred pharmaceutically acceptable salts of lercanidipine include may be either R or S enantiomers, or a racemic mixture thereof.

The term “modified release” means any type of release of the active ingredient, lercanidipine, from the composition of the present invention resulting in release over an extended period of time sufficient to maintain therapeutically effective plasma levels over similarly extended time intervals and/or to modify other pharmacokinetic properties of the active ingredient. Preferably the release provides for therapeutic plasma concentrations of lercanidipine for a period of from about 20 to about 25 hours following administration to a patient, and an average minimum plasma concentration Cₘᵢₙ of lercanidipine greater than about 0.1 to about 0.4 ng/ml over the duration of the dosing interval.

The term “bioavailability” refers to the rate and extent to which the active ingredient or active moiety, e.g., lercanidipine, is absorbed from a drug product, i.e., bead, and becomes systematically available.

As used herein, the term “pharmaceutically acceptable” refers to a biologically or pharmacologically compatible for in vivo use, and preferably means approved by a regulatory agency of the Federal or a State government or listed in the U.S. Pharmacopoeia or other generally recognized pharmacopoeia for use in animals, and more particularly in humans.

The terms “treat” and “treating” refer to reducing or relieving hypertension, e.g., decreasing either systolic or diastolic blood pressure in a patient by at least 10 mm Hg.

As used herein, a “therapeutically effective amount” refers to the amount of an active agent sufficient to lower the blood pressure of a patient with hypertension, e.g., the blood pressure is decreased by at about 15 mm Hg for systolic pressure or by about 10 mm Hg for diastolic pressure. A therapeutically effective amount of the active agent may or may not decrease the blood pressure in a person that does not have hypertension or may not decrease blood pressure in all persons with hypertension. In a preferred embodiment, the active agent decreases a patient's blood pressure to below 140/90 mm Hg. Treatment of other pathologies, such as heart failure or arthrosclerosis is also specifically contemplated as per, e.g., U.S. Pat. Nos. 5,696,139 and 5,767,136.

All weights and weight ratios specified for lercanidipine and pharmaceutically acceptable salts thereof are based on the weight of a molar equivalent of the hydrochloride salt of lercanidipine.

Pharmaceutical Compositions

The modified release bead formulation of the present invention is designed to provide modified release of lercanidipine upon exposure of the bead to a use environment, such as gastrointestinal fluid. It has been discovered that modified release compositions comprising the poor solubility and low permeability drug, lercanidipine, may be obtained by coating (i) an immediate release core comprising an inert core, (ii) a first layer comprising (a) lercanidipine, (b) a surfactant and (c) a binder, and (d) optionally a second layer comprising a film coating, with (iii) an outer layer comprising at least one release modifying acrylic polymer, and optionally a film coating.

Immediate Release Core

The immediate release core of the present invention comprises (i) an inert core and (ii) a first layer comprising (a) lercanidipine, (b) a surfactant and (c) a binder and (d) optionally a second layer comprising a film coating. The immediate release core may include additional excipients to improve appearance, handling and processing properties and/or dissolution properties of the active ingredient. Additional excipients contemplated by the present invention include, but are not limited to, carriers, diluents, lubricants, glidants and/or anti-adherent agents.

The inert core may comprise any pharmaceutically acceptable material, including but not limited to inorganic or organic non-pareil seeds, such as those made from microcrystalline cellulose, sugar or starch. Preferably the inert core has a mean size from about 10 and about 80 mesh. Preferably the ratio of the mass of the inert core to the mass of lercanidipine is from about 5:1 to about 20:1 and more preferably from about 5:1 to about 15:1.

The inert core is coated with a first layer comprising lercanidipine, a solubility/permeability enhancing surfactant and a binder. In one preferred embodiment, the lercanidipine is lercanidipine hydrochloride. Additionally, lercanidipine may be present in either the crystalline or the amorphous form. Lercanidipine which is present in the crystalline form may be present in any polymorphic form or mixtures thereof, including those disclosed in U.S. Published Application Nos. 2003/0083355 and 2003/0069285 which are incorporated herein by reference. Preferred pharmaceutically acceptable polymorphs of lercanidipine are crystalline Form I and II. Additionally, lercanidipine may be amorphous or a mixture or amorphous and crystalline forms, wherein the crystalline can be of the same polymorph or a combination of two or more polymorphs. Additionally, lercanidipine may be present in one or both of its enantiomeric forms.

One skilled in the art will appreciate that the pharmaceutical composition of the present invention may include one or more forms of lercanidipine, e.g., different salt forms, amorphous forms or crystalline forms, in order to achieve the desired in vitro dissolution profile and/or the desired in vivo plasma concentration of lercanidipine. In one embodiment, one skilled in the art may combine crystalline lercanidipine forms (I) and (II) to achieve desired properties, based upon bioavailability studies described in US. Published Application 2003/0083355 (herein incorporated by reference) that found lercanidipine crystalline polymorph form (II) to have a higher bioavailability than lercanidipine crystalline polymorph Form (I). Studies have also indicated, however, that form (I) has a shorter time to maximum concentration attainable compared to form (II) and that form (II) has a slightly higher systemic exposure (AUCₒ₋ₜ) and a delayed time of maximum concentration (Tₘₐₓ), compared to Form (I). The novel present invention incorporates sufficient solubility/permeability enhancer surfactant that allows for the use of different polymorphs.

Preferably lercanidipine is present in an amount sufficient to render a therapeutic effect when the modified release composition of the present invention is administered to a patient. Lercanidipine may be present any amount from about 0.001 to about 0.2 mg per mg of the total composition, and more preferably from about 0.005 mg to about 0.15 mg per mg of the total composition and most preferably 0.01 mg about 0.1 mg per mg of the total composition.

In addition to lercanidipine, the first layer coating the inert core comprises a surfactant. Surfactants may be incorporated in the beads of the present invention to facilitate the wetting of lercanidipine and promote its adhesion to the inert core and/or binders. Surfactant may also be incorporated for the purpose of enhancing or modulating the solubility of lercanidipine in the environment of use.

Surfactants of the present invention include, but are not limited to anionic and non-ionic surfactants such as sodium lauryl sulfate, poloxamers (copolymers of polyoxyethylene and polyoxypropylene), natural or synthetic lecitins as well as esters of sorbitan and fatty acids, such as Span® (commercially available from Sigma-Aldrich Co., St. Louis, Mo.), esters of polyoxyethylenesorbitan and fatty acids, such as Polysorbates or Tween® (commercially available from Sigma-Aldrich Co.) polyoxyethylated hydrogenated castor oils, such as Cremophor® (commercially available from BASF, Mount Olive, N.J.), polyoxyethylene stearates, such as Myrj® (commercially available from Uniqema, New Castle, Del.) or any combinations of the said surfactants. Preferably the surfactant is a polysorbate and most preferably the surfactant is Polysorbate 80 (e.g., Tween® 80, commercially available from Sigma-Aldrich Co., St. Louis, Mo.) or Vitamin E TPGS (Eastman Chemical, Kingsport, Tenn.)

The amount of surfactant may be adjusted, so as to moderate the solubility, permeability and bioavailability of lercanidipine. Preferably the ratio of surfactant to lercanidipine on a mass basis is from about 0.001:1 to about 0.2:1, more preferably from about 0.005:1 to 0.1:1 and most preferably from about 0.01:1 to about 0.075:1.

The first layer coating the inert core further comprises a binder. Binders are incorporated in the beads of the present invention to facilitate the adhesion of lercanidipine to the inert core. Preferably, the binder does not interfere with or decrease the solubility of lercanidipine. Suitable binders include, but are not limited to, either individually or in combination, such binding agents and adhesives as sucrose; gelatin; glucose; starch; cellulose materials such as, but not limited to, methylcellulose and sodium carboxymethylcellulose; alginic acid and salts of alginic acid; magnesium aluminum silicate; polyethylene glycol; guar gum; polysaccharide acids; bentonites; polyvinylpyrrolidone (povidone); polymethacrylates; hydroxypropyl methylcellulose (HPMC); hydroxypropyl cellulose (Klucel™); ethyl cellulose (Ethocel™); pregelatinized starch (such as National™ 1511 and Starch 1500).

Preferably the binder comprises prehydroxypropylmethyl cellulose and most preferably Opadry™ (commercially available from Colorcon, Inc., West Point, Pa.). Preferably the ratio of binder to lercanidipine on a mass basis is from about 0.01:1 to about 1:1, more preferably from about 0.05:1 to 0.5:1 and most preferably from about 0.1:1 to about 0.3:1.

Optionally the immediate release core may comprise a second layer comprising a film coating to improve the durability, appearance and/or handling of the bead composition. Preferably the film coating does not interfere with the dissolution and/or pharmacokinetic properties of the bead composition of the present invention. Examples of film coatings contemplated by the present invention include, but are not limited to, those that include hydroxypropylmethyl cellulose and particularly Opadry™ Clear. However, any film-former known in the art may be used. If a film coating is to be applied to the immediate release core, the preferred ratio of film coating to lercanidipine is from about 0.01:1 to about 1:1, more preferably from about 0.03:1 to 0.5:1 and most preferably from about 0.05:1 to about 0.3:1.

Coating Immediate Release Bead to Yield Modified Release Bead

The immediate release core may be coated with a release modifying acrylic polymer to create the modified release bead of the present invention. The release modifying acrylic polymer is intended to slow the release of lercanidipine from the first layer, thereby providing the desired in vitro release rate or in vivo plasma concentrations of lercanidipine. Moreover, the acrylic polymer is intended to facilitate the dissolution of lercanidipine in the preferred environmental fluid, e.g., the gastric fluid.

In addition to regulating the release of lercanidipine, the release modifying acrylic polymer should be capable of producing a strong, continuous film that is smooth and elegant, capable of supporting pigments and other coating additives, inert and tack-free.

Any acrylic polymer which is pharmaceutically acceptable can be used for the purposes of the present invention. Acrylic polymers contemplated by the present invention, include, but are not limited to; acrylic acid and methacrylic acid copolymers, methacrylic acid copolymers, methyl methacrylate copolymers, ethoxyethyl methacrylates, cynaoethyl methacrylate, methyl methacrylate, copolymers, methacrylic acid copolymers, methyl methacrylate copolymers, methyl methacrylate copolymers, methyl methacrylate copolymers, methacrylic acid copolymer, aminoalkyl methacrylate copolymer, methacrylic acid copolymers, methyl methacrylate copolymers, poly(acrylic acid), poly(methacrylic acid, methacrylic acid alkylamine copolymer, poly(methyl methacrylate), poly(methacrylic acid) (anhydride), methyl methacrylate, polymethacrylate, methyl methacrylate copolymer, poly(methyl methacrylate), poly(methyl methacrylate) copolymer, polyacrylamide, aminoalkyl methacrylate copolymer, poly(methacrylic acid anhydride), and glycidyl methacrylate copolymers. Additionally, polymers, e.g., cellulose derivatives, include but are not limited to ethyl cellulose and HPMC (Synchron, Methocel).

Most preferably the acrylic polymer of the present invention is an acrylic resin lacquer used in the form of an aqueous dispersion. Acrylic polymers embodied by the present invention include those commercially available under several generic and brand names. One skilled in art will recognize these polymers available from Rohm Pharma (Piscataway, N.J.) under the Tradename Eudragit R®. In further preferred embodiments, the acrylic coating comprises a mixture of two acrylic resin lacquers, copolymer of Acrylic acid and methacrylic acid ester, commercially available from Rohm Pharma under the Tradenames, e.g., Eudragit® RL 30 D (permeable to highly permeable) and Eudragit® RS 30 D (poorly permeable).

Eudragit® RL 30 D and Eudragit® RS 30 D are copolymers of acrylic and methacrylic esters with a low content of quaternary ammonium groups, the molar ratio of ammonium groups to the remaining neutral (meth)acrylic esters being 1:20 in Eudragit® RL 30 D and 1:40 in Eudragit® RS 30 D. The mean molecular weight is about 150,000. The code designations RL (high permeability) and RS (low permeability) refer to the permeability properties of these agents. Eudragit® RL/RS mixtures are insoluble in water and in digestive fluids. However, coatings formed from the same are swellable and permeable in aqueous solutions and digestive fluids.

The Eudragit® RL/RS dispersions of the present invention may be mixed together such that the mixture ratio of Eudragit® RL to Eudragit® RS ultimately obtain a modified release formulation having a desirable dissolution profile and/or pharmacokinetic profile. Desirable controlled release formulations may be obtained, for instance, from a retardant coating derived from 100% Eudragit® RL; or mixtures having a ratio of Eudragit® RL to Eudragit® RS of between about 50:50 to about 95:5 and more preferably between about 80:20 and 90:10. In this invention, the brand names “Eudragit” are used for ease of understanding only, one skilled in art will appreciate that these polymers are available under generic and other brand names.

Preferably the amount of outer layer is applied in an amount sufficient to yield a modified release bead having the desired dissolution profile and/or pharmacokinetic profile. Most preferably the outer layer is applied, such that the beads have an average radius from about 10 mesh to about 140 mesh mm and most preferably from about 14 mesh to about 35 mesh mm.

One skilled in the art will appreciate that the rate of lercanidipine release from the modified release bead composition may be controlled by factors such as the composition and binder content of the immediate release core, the thickness and permeability of the release modifying acrylic polymer coating and the surface-to-volume ratio of the beads themselves. It will be appreciated by those skilled in the art that increasing the thickness of the coating will decrease the release rate, whereas increasing the permeability of the coating or the surface-to-volume ratio of the beads will increase the release rate. One skilled in the art will appreciate that the above mentioned factors may be adjusted such that the modified release composition of the present invention achieves the desired in vitro dissolution rate, and/or the in vivo plasma concentration of lercanidipine over time.

Moreover, it will be appreciated by those skilled in the art that the desired in vitro dissolution rate, and/or the in vivo plasma concentration of lercanidipine over time, may be obtained by selecting one or more forms of lercanidipine, i.e., selecting one or more salts forms, crystalline forms (including one or more polymorphic forms) or amorphous forms for use in the modified release beads of the present invention.

In one preferred embodiment of the present invention, an effective amount of a plasticizer may be included in the release modifying acrylic polymer layer, to improve the physical properties of the outer layer. Suitable plasticizers embodied by the present invention include, but are not limited to, citric acid esters such as triethyl citrate, tributyl citrate, dibutyl phthalate, and possibly 1,2-propylene glycol, polyethylene glycols, propylene glycol, diethyl phthalate, castor oil, and triacetin. In one preferred embodiment the acrylic polymer forming the outer layer includes triethyl citrate as a plasticizer.

Plasticizers may be incorporated into the outer layer in any amount sufficient to impart the modified release composition of the present invention with the desired physical properties. Preferably the plasticizer is present in amounts between about 10 and about 35% and most preferably between about 15 and about 25% of the polymer weight. One skilled in the art, however, will appreciate that the precise amount of plasticizer may depend upon several factors including the type of polymer and the coating conditions and in many instances may require routine experimentation to determine.

The outer layer of the modified release compositions of the present invention may include, in addition to a release modifying acrylic polymer, a film coating. The optional film coating may be added to improve the durability and appearance of the bead. Preferably the film coating does not interfere with the release of lercanidipine from the first layer when the bead is exposed to its environment of use. Preferably the film coating present in the outer layer is hydroxypropylmethyl cellulose and most preferably Opadry™ Clear. However, any film-former known in the art may be used. Film coating may be incorporated into the outer layer in any amount sufficient to impart the modified release composition of the present invention with the desired physical properties. Preferably the film coating is present in amount between about 1 and about 5% and most preferably between about 2 and about 3% of the weight of the bead composition.

Optionally, the pharmaceutical compositions of the present invention may include additional excipients to improve appearance, handling and processing properties and/or dissolution properties of the active ingredient. Additional excipients contemplated by the present invention include, but are not limited to, carriers, diluents, lubricants, glidants and/or anti-adherent agents.

Suitable lubricants and/or glidants include, but are not limited to, either individually or in combination, such lubricants and/or glidants as glyceryl behenate (Compritol™ 888); metallic stearates (e.g., magnesium, calcium and sodium stearates); stearic acid; hydrogenated vegetable oils (e.g., Sterotex™); talc; waxes; Stearowet™; boric acid; sodium benzoate and sodium acetate; sodium chloride; DL-Leucine; polyethylene glycols (e.g., Carbowax™ 4000 and Carbowax™ 6000); sodium oleate; sodium benzoate; sodium acetate; sodium lauryl sulfate; sodium stearyl fumarate (Pruv™); and magnesium lauryl sulfate.

Additional suitable anti-adherents or glidants include, but are not limited to, either individually or in combination, such anti-adherents as talc, cornstarch, DL-Leucine, sodium lauryl sulfate, and metallic stearates.

Other carrier materials (such as colorants, flavors and sweeteners) and modes of administration are known in the pharmaceutical art and can be used in the preparation of the pharmaceutical compositions of the present invention.

Manufacture of Pharmaceutical Compositions

The modified bead composition of the present invention may be manufactured using any number of processes well known in the art. In one embodiment the composition of the present invention may be prepared as a bead by first forming an immediate release core by coating an inert core with an aqueous suspension containing lercanidipine. The immediate release core may then be coated with an outer coating comprising a release modifying acrylic polymer to prepare the modified release composition of the present invention. Optionally, a film coating may be applied over the release modifying acrylic polymer to enhance the durability and appearance of the bead.

In one embodiment, inert cores are preheated in a fluidized bed coated (e.g., GPGC5, Glatt Air Technique, Ramsey N.J.), for about 10 minutes and more preferably for about 5 minutes, between about 30° C. and about 45° C. and more preferably between about 35° C. and 40° C. Drug loading may be carried out using any method known in the art, such spray coating, although other coating methods may be used. Preferably, the inert cores are coated with a suspension containing lercanidipine, a binder, a surfactant and purified water in a fluidized bed reactor using a spray pressure between about 1 and 3 bars, at a temperature between 30° C. and about 45° C. and more preferably between about 35° C. and 40° C.

Drug loaded beads may optionally be film coated by coating the beads with an aqueous dispersion of material such as Opadry™ Clear. An aqueous film coating dispersion may be applied using any method known in the art, such as spray coating the beads in a fluidized bed coater at a spray pressure between about 1 and 3 bars and a temperature between about 30° C. and about 45° C. and more preferably between about 35° C. and 40° C.

Following drug loading and/or film coating, the cores may be dried and cured. In one embodiment, the immediate release cores are dried in a fluidized bed for about 10 minutes and more preferably for about 5 minutes, between about 30° C. and about 45° C. and more preferably between about 35° C. and 40° C.

An aqueous suspension containing the release modifying acrylic polymer may be applied to the immediate release core by spraying, using any suitable spray equipment known in the art. An aqueous suspension containing the release modifying acrylic polymer may be prepared by dissolving the polymer in water or in an organic solvent or mixture of organic solvents. Useful organic solvents for this purpose are acetone, isopropyl alcohol, and methylene chloride. The aqueous suspension may also include a plasticizer. Useful plasticizers include citric acid esters such as triethyl citrate, tributyl citrate, dibutyl phthalate, and possibly 1,2-propylene glycol, polyethylene glycols, propylene glycol, diethyl phthalate, castor oil, and triacetin. The plasticizer may be present in an amount from about 10 to about 35% based upon the weight of the polymers.

Following coating the beads may be cured and dried. One skilled in the art will appreciate that drying and curing conditions will vary depending upon several factors including for example, the size of the substrate, the thickness of the coating, and the amount of hydrophobic material in the composition. In one embodiment, the immediate release cores are dried in a fluidized bed for about 10 minutes and more preferably for about 5 minutes, between about 30° C. and about 45° C. and more preferably between about 35° C. and 40° C. The bead compositions of the present invention may be cured and dried following manufacture. One skilled in the art will appreciate that drying and curing conditions will vary depending upon several factors including for example, the size of the substrate, the thickness of the coating, and the amount of hydrophobic material in the composition. Preferably, the modified release compositions of the present invention are cured in an oven or other suitable devise at about between 40° C. and 60° C. and more preferably about 50° C. for between about 8 and about 48 hours. Unit dosage forms

To form oral unit dosage forms, dried beads may be combined and loaded into gelatin capsules, or other delivery devices suitable for oral administration. Preferably the unit dosage forms comprise a sufficient amount of the modified release beads of the present invention to impart a therapeutic effect when the dosage form is administered to a patient. More preferably the unit dosage form comprises from about 2 to about 80 mg of lercanidipine, and most preferably about 5 to about 80 mg of lercanidipine.

In one embodiment the modified release compositions of the present invention may be combined with an immediate release composition and/or another modified release composition and/or pulsatile pH dependent modified release compositions to yield a unit dosage exhibiting a multi-phase release profile. Preferably the unit dosage form comprises both immediate and modified release compositions and provides a total dosage of lercanidipine of about 2 to about 80 mg of and more preferably about 5 to about 80 mg, wherein from about 5 to about 80 mg of the lercanidipine is provided as a modified release composition.

Preferably the ratio of immediate release to modified release compositions is such that the dosage form, when administered to a patient, provide both rapid and longer term relief from hypertension. Preferably, the ratio of immediate to modified release compositions is such that the dosage form provide for maximum plasma concentration of lercanidipine from about 10 to about 14 ng/mL and therapeutic plasma concentrations of lercanidipine for a period of about 20 to about 25 hours. In one embodiment, the time to reach maximum plasma concentration, Tₘₐₓ, is about 1 to 12 hours. In one embodiment the ratio of immediate release to modified release compositions is preferably from about 1:1 to 1:50, more preferably from about 1:2 and 1:20 and most preferably from about 1:5 to 1:10.

Dissolution Profile

The modified release compositions of the present invention are designed to provide sustained release of lercanidipine over the duration of the dosing interval. To ensure that the modified release compositions provide the desired effect in vitro, e.g., therapeutic plasma concentrations for a period of about 20 to about 25 following administration to a patient, it is first necessary to establish desired in vitro dissolution properties. Although the mere presence of a substance in the gastrointestinal fluid is not itself sufficient to ensure bioavailability, the dissolution properties of an active agent are nonetheless important in evaluating its ability to be absorbed and made available at the site of action. Therefore, when evaluating the potential bioavailability of an active agent is important to determine its dissolution profile.

The dissolution profile for an active agent from a dosage unit is determined as the proportion of the amount of active agent released from the dosage unit over a specified time. The test method used references the results, so it is important to specify the method as well as the conditions under which measurements were made. Preferably the dissolution properties of the modified release compositions of the present invention are determined using the dissolution method, USP I basket method, 100 RPM, 900 ml, aqueous buffer (0.01 N HCl), for at 37° C.

With the above in mind, in one embodiment the in vitro dissolution of lercanidipine at various time points for compositions in accordance with the present invention is preferably about 80% dissolved within about 3 to 12 hours. more preferably at least about 80% dissolved within about 6 hours, and still more preferably at least about 15% dissolved within about 1 hour.

In still another embodiment, the in vitro dissolution of lercanidipine at various time points for compositions in accordance with the present invention is preferably about 80% dissolved in vitro within about 12 hours, and still more preferably at least about 25% dissolved within about 1 hour. One skilled in the art will appreciate that the dissolution rates can be adjusted to release more than 80% of the drug from 2 to 24 hours based on the compositions and processing.

Pharmacokinetic Profiles

In addition to providing for modified dissolution of lercanidipine in an use environment, e.g., gastric fluid, it is an objective of the present invention to provide a modified release composition having a pharmacokinetic profile which provides for sustained relief of symptoms associated with hypertension, while avoiding undesirable side effects. Such a pharmacokinetic profile provides for a gradual rise in lercanidipine plasma concentration to therapeutic levels following administration to a patient, e.g., from about 8 to about 12 ng/ml of lercanidipine, followed by a steady decline in plasma concentration to a level greater than about 0.1 to 0.4 ng/ml of lercanidipine. Preferably, the pharmacokinetic profile does not have any erratic peaks or troughs, but rather provides for a steady and consistent rise in lercanidipine concentration to therapeutic levels, followed by a steady and consistent decline.

Additionally, it is an objective of the present invention to provide modified release composition which provides for sustained, e.g., long term, plasma concentration of lercanidipine at therapeutic levels. Preferably, upon administration of the modified release composition of the present invention to a patient the composition provides for sustained therapeutic plasma concentrations of lercanidipine for about 20 to about 25 hours.

Treatment of Specific Conditions and Disorders

The pharmaceutical composition or unit dosage forms of the present invention may be administered to an animal, preferably a human being, in need of antihypertensive treatment. The pharmaceutical composition or unit dosage form of the present invention may be administered according to a dosage and administration regimen defined by routine testing in light of the guidelines given above in order to obtain optimal antihypertensive activity and a decrease in blood pressure while minimizing toxicity or side-effects for a particular patient. However, such fine turning of the therapeutic regimen is routine in light of the guidelines given herein.

The dosage of the modified release composition of the present invention may vary according to a variety of factors such as underlying disease state, the individual's condition, weight, sex and age and the mode of administration. For oral administration, the pharmaceutical compositions can be provided in the form of scored or unscored unit dosage forms.

In one embodiment for the treatment of hypertension, the pharmaceutical composition or oral dosage form comprising modified release beads of the present invention preferably comprises from about 2 to 80 mg lercanidipine. More preferably, the composition or dosage form comprises from about 5 to 80 mg lercanidipine.

The pharmaceutical composition or unit dosage form may be administered in a single daily dose, or the total daily dosage may be administered in divided doses. In addition, co-administration or sequential administration of other active agents may be desirable. The modified release bead compositions of the invention may be combined with any known drug therapy, preferably for treatment of hypertension. For example, bimodal therapy involving a diuretic, a Preceptor blocker, an ACE inhibitor or an angiotensin II receptor antagonist is contemplated by the present invention (see, e.g., U.S. patent application Ser. No. 10/791,148, which is hereby incorporated by reference.)

The lercanidipine formulation of the current invention may be combined with additional active agents. Two different 1,4-dihydropyridines may be used, or lercanidipine may be combined with other active agents or other therapies For example, a lercanidipine formulation may be combined with an ACE inhibitor, such as enalapril, described in U.S. Patent Publication No. 2003/0180355, or with lisinopril as described in commonly-owned U.S. patent application Ser. Nos. 10/688,061 and 10/829,932. Lercanidipine may also be combined with an angiotensin II receptor blocker (ARB) such as irbesartan or olmesartan (U.S. patent application Ser. No. 10/791,148). Also contemplated by the present invention is addition of a diuretic or a receptor blocker to the lercanidipine formulation. Exemplary diuretics include thiazide diuretics, potassium sparing diuretics, or loop diuretics, such as hydrochlorothiazide, spironolactone, and ethacrynic acid, respectively.

Non-limiting examples of ACE inhibitors include benazepril, captopril, cilazapril, enalapril, fentiapril, fosinopril, indopril, lisonopril moexipril, perindopril, quinapril, ramipril, spirapril and their pharmaceutically acceptable salts. Exemplarily diuretics include furosemide, hydrochlorothiazide, torasemide, indapamide and eplerenone. Exemplarily CCB's include amlodipine, nifedipine and verapamil. Exemplarily beta blockers include atenolol, carvediol, nadolol and propranolol. Exemplarily alpha blockers include clonidine and prazosin. Exemplarily ARBs include, candesartan, eprosartan, irbesartan, losartan, olmesartan, saprisartan, telmisartan, valsartan, E-1477, SC-52458, ZD-8731, and their pharmaceutically acceptable salts. Additional ARBS include those found in EP 443983, EP 253310, EP 403159, EP 420237, EP 502314, EP 504888, EP 514198, EP 475206, and WO 93/20816, each of which are herein incorporated by reference.

The lercanidipine formulations may also be combined in a therapy with a second active agent, such as those described above, where the two agents are administered sequentially. Either the lercanidipine or the second agent may be delivered first, and the time between treatment of the lercanidipine and second agent may be for a period from about 1-2 hours, to about 2-6 hours, to about 6-12 hours, or to about 12-24 hours following administration of the first agent. Similarly, this same time period may occur between a first and third agent in the case of a three-way combination. Alternatively, simultaneous administration of the 1,4-dihydropyrine and second active agent, with or without sequential administration of either the 1,4-dihydropyrine and second active agent could also be employed.

For combination therapy the compounds may initially be provided as separate dosage forms until an optimum dosage combination and administration regimen is achieved. Therefore, the patient may be titrated to the appropriate dosages for his/her particular hypertensive condition. After the appropriate dosage of each of the compounds is determined to achieve a decrease in blood pressure without untoward side effects, the patient then may be switched to a single dosage form containing the appropriate dosages of each of the active agents, or may continue with a dual dosage form.

The exact dosage and administration regimen utilizing the combination therapy of the present invention is selected in accordance with a variety of factors including type, species, age, weight, sex and medical condition of the patient; the severity and etiology of the hypertension to be treated; the route of administration; the renal and hepatic function of the patient; the treatment history of the patient; and the responsiveness of the patient. Optimal precision in achieving concentrations of compounds within the range that yields efficacy without toxicity requires a regimen based on the kinetics of the drug's availability to target sites. This involves a consideration of the absorption, distribution, metabolism, excretion of a drug, and responsiveness of the patient to the dosage regimen. However, such fine tuning of the therapeutic regimen is routine in light of the guidelines given herein.

### EXAMPLES

The following examples of pharmaceutical bead compositions and methods of making the same are now disclosed. The following examples are illustrative in nature of the various aspects of the present invention and are not intended to be limiting in any manner.

### Example 1

### Preparation of Lercanidipine Immediate Release Core

The present examples describes the composition and manufacture of an immediate release core. The composition of the immediate release core is shown in Table 1 below. All weights are provided on the basis of the mass of the dried bead composition.

TABLE 1
Lercanidipine immediate release cores

| Ingredient | mg/g | Weight % Composition |
|---|---|---|
| Lercanidipine HCl | 122.6 | 12.26 |
| Polysorbate 80, NF | 9.2 | 0.92 |
| Sugar Spheres, USP | 818 | 81.80 |
| Opadry ™ Clear (Binder) | 30.6 | 3.06 |
| Opadry ™ Clear (Film | 19.6 | 1.96 |
| Coating) | | |

The lercanidipine immediate release core of the present example was prepared by loading approximately 8.18 kg sugar spheres, USP Pauler Crop, Cranbury N.J. having a size of approximately 20-25 mesh into a GPCG5 fluidized bed coater. The sugar spheres were preheated for about 5 minutes between 34 and 44° C.

The preheated spheres were spray coated with an aqueous lercanidipine suspension in a GPCG5 fluidized bed coated, using a Wuster System [Glatt Air Technique, Ramsey N.J.] at a spraying pressure between 1 and 3 bars and a temperature between 34 and 44° C.

The lercanidipine suspension was prepared by first preparing a suspension of Opadry™ Clear by mixing 0.306 Kg Opadry™ Clear (Colorcon, Inc., West Point, Pa.) in 11.6 L purified water with continuous stirring until fully dissolved. The suspension of Opadry™ Clear was divided into equal halves. To one half 0.092 Kg Polysorbate 80 Spectrum Chemical, New Brunswick, N.J. was added with continuous stirring followed by the addition of 1.226 Kg lercanidipine HCl (Recordati SpA, Milan, Italy). Once the lercanidipine HCl was fully dispersed, the second half of the Opadry™ Clear was added to complete the solution.

Following drug loading the beads were film coated by coating with Opadry™ Clear. A aqueous dispersion of Opadry™ Clear was prepared by mixing 01.96 Kg Opadry™ Clear with 2.45 L purified water with continuous stirring until the Opadry™ Clear was completely dissolved. The film coating solution was applied by spraying the beads in a fluidized bed coater using a spray pressure between about 1 and 3 bars, at a temperature between about 34 and 44° C.

Film coated beads were dried in a fluidized bed for about 5 minutes between about 34 and 44° C. Beads were mixed in a V-blender and stored sealed under suitable conditions.

### Example 2

### Preparation of Lercanidipine Modified Release Beads, Type III

The present example describes the composition and manufacture of a lercanidipine modified release bead release modifying acrylic polymer coating member comprising Eudagrit® RL 30D and Opadry™ Clear, and trietyl citrate as a plasticizer. The release modifying acrylic polymer coating was applied to the immediate release core described in Example 1 to achieve the modified release composition of the present Example. The composition of the present modified release bead is shown in Table 2 below.

TABLE 2
Type III, lercanidipine modified release bead

| Ingredient | Mass (g) | Weight % Composition |
|---|---|---|
| Lercanidipine IR core | 1800 | 89.1 |
| Eudragrit ® RL 30D | 300 | 4.5 |
| Opadry ™ Clear | 13.5 | 0.7 |
| Triethyl Citrate, PG/NF | 18 | 0.9 |
| Talc, USP | 58.5 | 2.9 |
| Talc, USP, after coating | 39.6 | 2.0 |
| mixture | | |

A fraction of the immediate release cores, prepared as described in Example 1 above were loaded into a fluid bed coater (Glatt Air Technique, Ramsey NJGPCG3) and heated at between about 26 to 36° C. for about five minutes. The preheated cores were then coated with an aqueous suspension consisting of Eudagrit® RL 30D, triethyl citrate and up to about 15% (w/w Opadry™ Clear, and talc. The cores were coated to a total weight gain of about 10%. Following coating the beads were cured by drying in an oven at 50° C. for 24 hours. For comparison purposes three batches of Type III beads were prepared as described herein and subjected to dissolution analysis as described below.

Following curing, the modified release beads of the present example were subjected to dissolution analysis. Dissolution analysis was carried out via the USP I basket method, in 900 ml, aqueous buffer (0.01 N HCl), for 8 hours at 37° C., 100 RPM. F2 values were calculated from the data points as follows:
F2=50×log{[1+(1/n)Σₜ₋₁ⁿ(Rₜ−Tₜ)²]⁻0.5×100}

t—dissolution time point

n—number of time points tested

Rₜ—reference batch dissolution time (t)

Tₜ—test batch dissolution at time (t)

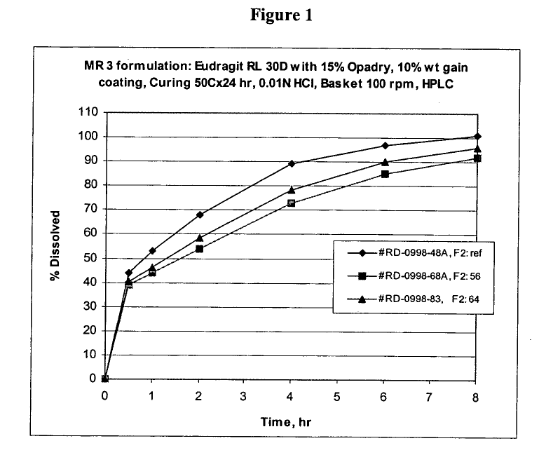

The dissolution results for the three batches of Type III beads are set forth in Table 3 below and are depicted in FIG. 1. The dissolution profiles for each of the three batches of Type III were compared using a model independent statistical approach and the similarity factor, F2. F2 values of 50 or greater ensure equivalence of the two curves. Compared to the reference curve (batch I beads), the F2 values for batches II and III were 56 and 64 respectively.

TABLE 3
Dissolution % Released data for Type III modified release beads

| Time (Hr) | Batch I | Batch II | Batch III |
|---|---|---|---|
| 0.5 | 44 | 39 | 40 |
| 1 | 53 | 44 | 46 |
| 2 | 68 | 54 | 59 |
| 4 | 89 | 73 | 78 |
| 6 | 97 | 85 | 90 |
| 8 | 100 | 92 | 96 |

### Example 3

### Preparation of Lercanidipine Modified Release Beads, Type IV

The present example describes the composition and manufacture of a lercanidipine modified release bead in which a mixture of Eudragit® RL 30D and Eudragit® RS 30D was applied as an outer coating member to the immediate release core described in Example 1. The composition of the modified release bead of the present Example is shown in Table 4 below.

TABLE 4
Type I, lercanidipine modified release bead Formulation Type IV

| Ingredient | Mass (g) | Weight % Composition |
|---|---|---|
| Lercanidipine IR core | 1800 | 88.2 |
| Eudragrit ® RL 30D | 270 | 4.0 |
| Eudragrit ® RS 30D | 30 | 1.5 |
| Triethyl Citrate, PG/NF | 18 | 0.9 |
| Talc, USP | 72 | 3.5 |
| Talc, USP, after coating | 39.6 | 1.9 |
| mixture | | |

A fraction of the immediate release cores, prepared as described in Example 1 above were loaded into a fluid bed coater (Glatt Air Technique, Ramsey N.J. GPCG3) and heated at between about 26 and 36° C. for about five minutes. The preheated cores were then coated with an aqueous suspension containing a mixture of Eudagrit® RL 30D and Eudagrit® RS 30D in a ratio of 90:10, triethyl citrate and talc. The cores were coated to a total weight gain of about 10%. Following coating the beads were cured by drying in an oven at 50° C. for 24 hours. For comparison purposes three identical batches of Type II beads were prepared as described herein and subjected to dissolution analysis as described below.

Following curing, the modified release beads of the present example were subjected to dissolution analysis. Dissolution analysis was carried out via the USP I basket method, in 900 ml, aqueous buffer (0.01 N HCl), for 14 hours at 37° C., 100 RPM. F2 values were calculated from the data points as follows:
F2=50×log{[1+(1/n)Σₜ₋₁ⁿ(Rₜ−Tₜ)²]^{-0.5}×100}

t—dissolution time point

n—number of time points tested

Rₜ—reference batch dissolution time (t)

Tₜ—test batch dissolution at time (t)

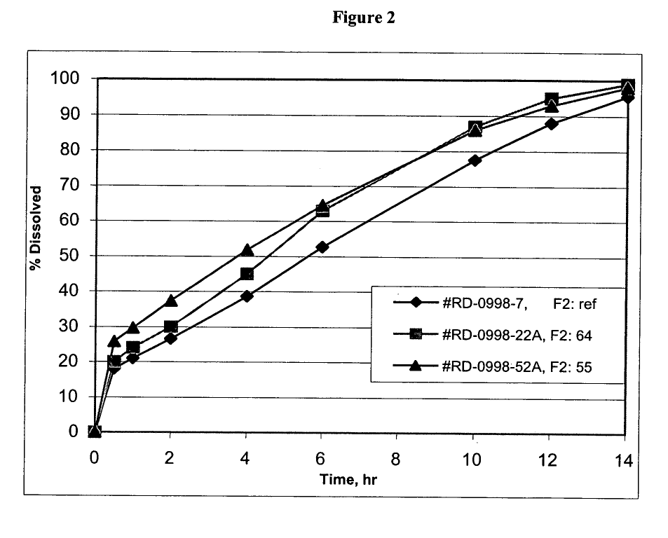

The dissolution results for the three batches of Type IV beads are set forth in Table 5 below and are depicted in FIG. 2. The dissolution profiles for each of the three batches of Type IV were compared using a model independent statistical approach and the similarity factor, F2. F2 values of 50 or greater ensure equivalence of the two curves. Compared to the reference curve (batch I beads), the F2 values for batches II and III were 64 and 55 respectively.

TABLE 5
Dissolution % Released data for Type IV modified release beads

| Time (Hr) | Batch I | Batch II | Batch III |
|---|---|---|---|
| 0.5 | 18 | 20 | 26 |
| 1 | 21 | 24 | 30 |
| 2 | 27 | 30 | 37 |
| 4 | 39 | 45 | 52 |
| 6 | 53 | 63 | 65 |
| 10 | 78 | 87 | 86 |
| 12 | 88 | 95 | 93 |
| 14 | 96 | 99 | 98 |

### Example 4

### Preparation of additional Formulations of Lercanidipine Modified Release Beads Type III

The present Example describes the additional formulations of lercanidipine modified release beads, Type III. Modified release of the present Example were prepared, substantially as described in Example 2, however, the amount of Opadry™ Clear in the outer layer was varied in order to moderate the dissolution of lercanidipine. Modified release beads having the composition described in Table 6 below were prepared and subjected to dissolution analysis.

TABLE 6
Additional formulations of Type III,
lercanidipine modified release beads

| | Type III, | Type III, | Type III, | Type III |
|---|---|---|---|---|
| | formulation | formulation | formulation | formulation |
| Ingredient | II | III | IV | V |
| Lercanidipine | 1800 | 1800 | 1800 | 1800 |
| IR core | | | | |
| Eudagrit ® | 300 | 300 | 300 | 300 |
| RL 30D | | | | |
| Opadry ™ | 0 | 4.5 | 9 | 13.5 |
| Clear | | | | |
| Triethyl | 18 | 18 | 18 | 18 |
| Citrate, | | | | |
| PG/NF | | | | |
| Talc, USP | 72 | 67.5 | 63 | 58.5 |
| Talc, USP | 39.6 | 39.6 | 39.6 | 39.6 |
| after | | | | |
| coating | | | | |
| mixture | | | | |

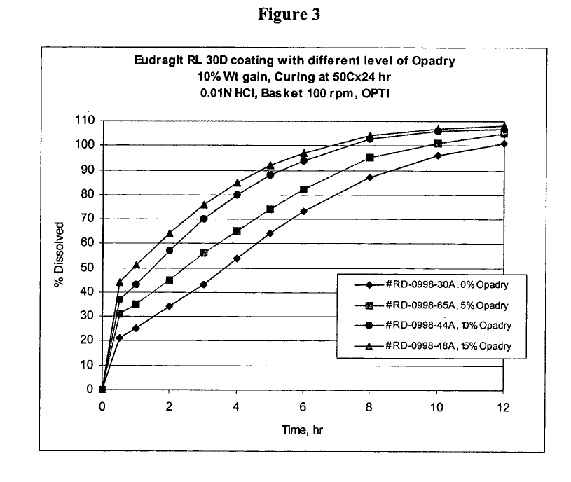

Modified release beads having the composition described in Table 6, were prepared as described as described in Example 2. The beads were cured in an oven at 50° C. for 24 hours. Following curing, the modified release beads of the present Example were subjected to dissolution analysis. Dissolution analysis was carried out via the USP I basket method, in 900 ml, aqueous buffer (0.01 N HCl containing Polysorbate 80), for 14 hours at 37° C., 100 RPM. The results of the dissolution analysis are set forth in Table 7, below, and and depicted in FIG. 3.

TABLE 7
Dissolution % Released data for formulations
of Type III modified release beads

| Time | | | | |
|---|---|---|---|---|
| (Hr) | Formulation II | Formulation III | Formulation IV | Formulation V |
| 0.5 | 21 | 31 | 37 | 44 |
| 1 | 25 | 35 | 43 | 51 |
| 2 | 34 | 45 | 57 | 64 |
| 4 | 54 | 65 | 80 | 85 |
| 6 | 73 | 82 | 94 | 97 |
| 10 | 96 | 100 | 100 | 100 |
| 12 | 100 | 100 | 100 | 100 |

### Example 5

### Preparation of Additional Formulations of Lercanidipine Modified Release Beads, Type IV

The present Example describes the additional formulations of lercanidipine modified release beads, Type IV. Modified release of the present Example were prepared, substantially as described in Example 3, however, the ratio of Eudagrit® RL 30D to Eudagrit® RS 30D in the outer layer was varied in order to moderate the dissolution of lercanidipine. Modified release beads having the composition described in Table 8 below were prepared and subjected to dissolution analysis.

TABLE 8
Additional formulations of Type IV, lercanidipine modified release beads

| | Formulation | Formulation | Formulation | Formulation | Formulation | Formulation |
|---|---|---|---|---|---|---|
| Ingredient | II | III | IV | V | VI | VII |
| Lercanidipine IR core | 1800 | 1800 | 1800 | 1800 | 1800 | 1800 |
| Eudagrit ® RL 30D | 300 | 270 | 240 | 210 | 180 | 150 |
| Eudagrit ® RS 30D | 0 | 30 | 60 | 90 | 120 | 150 |
| Triethyl Citrate, PG/NF | 18 | 18 | 18 | 18 | 18 | 18 |
| Talc, USP | 72 | 72 | 72 | 72 | 72 | 72 |
| Talc, USP after coating mixture | 39.6 | 39.6 | 39.6 | 39.6 | 39.6 | 39.3 |

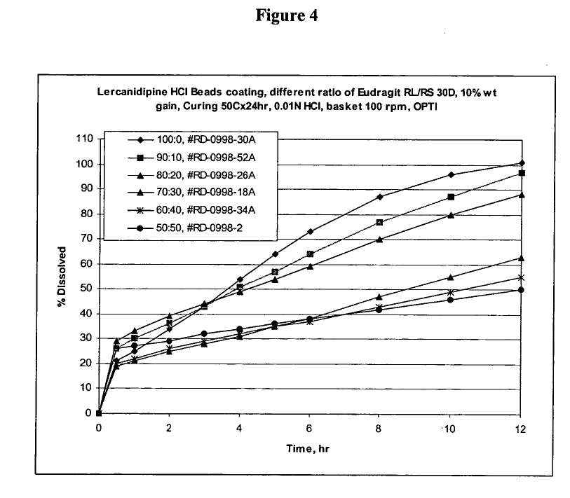

Modified release beads having the composition described in Table 8, were prepared as described in Example 3. The beads were cured in an oven at 50° C. for 24 hours. Following curing, the modified release beads of the present Example were subjected to dissolution analysis. Dissolution analysis was carried out via the USP I basket method, in 900 ml, aqueous buffer (0.01 N HCl containing Polysorbate 80), for 12 hours at 37° C., 100 RPM. The results of the dissolution analysis are set forth in Table 9, below, and depicted in FIG. 4.

TABLE 9
Dissolution data for formulations
of Type IV modified release beads

| | Form. | Form. | Form. | Form. | Form. | Form. |
|---|---|---|---|---|---|---|
| Time | II | III | IV | V | VI | VII |
| 0.5 | 21 | 26 | 29 | 19 | 20 | 26 |
| 1 | 25 | 30 | 33 | 21 | 22 | 27 |
| 2 | 34 | 36 | 39 | 25 | 26 | 29 |
| 4 | 54 | 51 | 49 | 31 | 32 | 34 |
| 6 | 73 | 64 | 59 | 38 | 37 | 38 |
| 10 | 96 | 87 | 80 | 55 | 49 | 46 |
| 12 | 101 | 97 | 88 | 63 | 55 | 50 |

### Example 6

### Preparation of Unit Dosage Form Comprising Immediate and Modified Release Lercanidipine Beads

Lercanidipine modified release beads, prepared as described in Examples 2 and 3 above, were combined with lercanidipine immediate release beads to form a solid dosage form. The modified release beads of the present example were prepared having the composition shown in Table 10. Lercanidipine immediate release beads of the present example were prepared having the composition shown in Table 11. The immediate release beads were prepared according to the method described in Example 1 for the preparation of immediate release cores.

TABLE 10
Lercanidipine modified release beads

| | Type III | Type IV |
|---|---|---|
| Ingredient | Formulation VI | Formulation VIII |
| Lercanidipine IR core | 1800 | 1800 |
| Eudagrit ® RL 30D | 900 | 810 |
| Eudagrit ® RS 30D | 0 | 90 |
| Triethyl Citrate, PG/NF | 54 | 54 |
| Talc, USP | 175.5 | 216 |
| Opadry Clear | 40.5 | 0 |
| Talc, USP after coating | 40 | 40 |
| mixture | | |

TABLE 11
Lercanidipine immediate beads

| Ingredient | Mass (mg) | Weight % Composition |
|---|---|---|
| Lercanidipine HCl | 102.12 | 12.26 |
| Polysorbate 80, NF | 7.66 | 0.92 |
| Sugar Spheres, USP | 681.6 | 81.80 |
| Opadry ™ Clear (Binder) | 25.5 | 3.06 |
| Opadry ™ Clear (Film | 16.3 | 1.96 |
| Coating) | | |

Two separate unit dosage forms were prepared, one containing Type III modified release beads (prepared as described in Example 2) and another containing Type IV modified release beads (prepared as described in Example 3). The two unit dosage forms, termed Prototype III and Prototype IV were prepared as described in Table 12 below.

TABLE 12
Unitary solid dosage forms

| Proto- | Type of modified | Amount of modified | Amount of immediate |
|---|---|---|---|
| type | release bead | release bead (mg) | release bead (mg) |
| III | Type III | 183.1 | 81.6 |
| IV | Type IV | 183.1 | 81.6 |

Both prototype III and IV were subjected to dissolution analysis. The dissolution analysis was carried out via the USP I basket method, in 900 ml, aqueous buffer (0.01 N HCl), at 37° C., 100 RPM. For prototype III, dissolution analysis was carried out for 8 hours and for prototype IV, dissolution analysis was carried out for 14 hours.

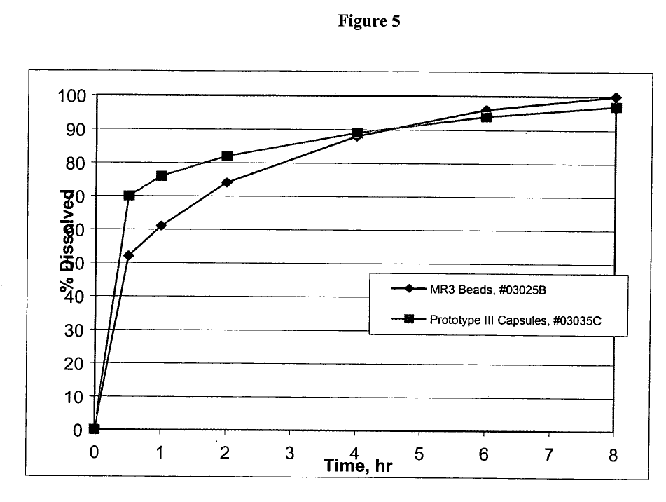

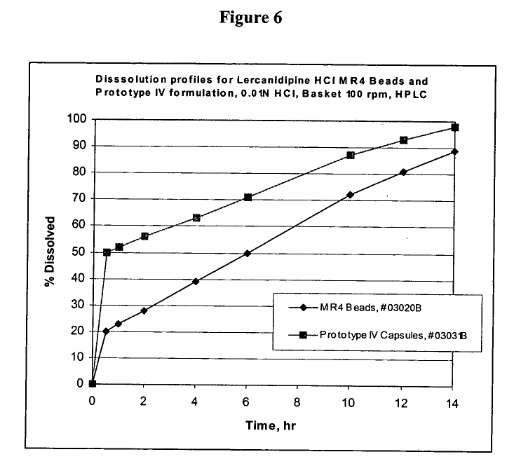

The results of the dissolution analysis for prototypes III and IV are shown in FIGS. 5 and 6, respectively. For comparative purposes, FIGS. 5 and 6 also show the dissolution profile of modified release bead compositions type III and type IV. From the dissolution profiles, it is evident that both prototypes III and IV provide both immediate and modified release of lercanidipine in vitro. The dissolution profiles of prototypes III and IV demonstrate that about 50% of the lercanidipine is dissolved within about 2 hours. The dissolution profiles as demonstrate that the prototypes provide modified release of lercanidipine in vitro. About 97% of the lercanidipine was dissolved from prototype III within about 8 hours, while about 98% of the lercanidipine was dissolved from prototype IV within about 14 hours.

The present invention is not to be limited in scope by the specific embodiments described herein. Indeed, various modifications of the invention in addition to those described herein will become apparent to those skilled in the art from the foregoing description and the accompanying figures. Such modifications are intended to fall within the scope of the appended claims.

It is further to be understood that all values are approximate, and are provided for description.

Patents, patent applications, publications, product descriptions, and protocols are cited throughout this application, the disclosures of which are incorporated herein by reference in their entireties for all purposes.

What is claimed is:

1. A modified release solid dosage form comprising lercanidipine, wherein upon entry of the dosage form to an use environment more than about 80% of the lercanidipine is released within about 3 to 12 hours and wherein the average Tₘₐₓ is within the range from about 2 hour to about 12 hours.

2. The modified release solid dosage form according to claim 1, wherein lercanidipine is lercanidipine hydrochloride.

3. The modified release solid dosage form according to claim 1, wherein lercanidipine is present in amounts ranging from about 2 mg to about 80 mg per unit dose.

4. The modified release solid dosage form of claim 1, wherein the solid dosage form is encapsulated within a capsule.

5. The modified release solid dosage form of claim 1, wherein the solid dosage form is compressed into a tablet.

6. The modified release solid dosage form of claim 1, wherein the dosage form is suitable for once daily oral administration.

7. The modified release solid dosage form of claim 1, wherein the dosage form is suitable for twice daily oral administration.

8. The modified release solid dosage form according to claim 1 wherein the dosage form is administered to a mammal in need thereof.

9. The modified release solid oral dosage form according to claim 8, wherein the mammal is a human.

10. A method of treating hypertension in a patient in need thereof comprising administering the modified release solid dosage form of claim 1.

11. The method of claim 10, wherein the average maximum plasma concentration of the lercanidipine is from about 0.5 to about 12 ng/ml, per 20 mg dose of lercanidipine, for a period from about 20 to 25 hours following administration to a patient.

12. A modified release pharmaceutical composition comprising:
(1) a core comprising of at least lercanidipine, and optionally a second layer comprising a film coating; and
(2) an outer-most layer comprising at least one release modifying polymer, and optionally a second layer comprising a film coating,
wherein the modified release pharmaceutical composition has an in vitro dissolution profile wherein more than about 80% of the lercanidipine is released within about the first 6 to 12 hours following entry of the pharmaceutical composition into an use environment.

13. The pharmaceutical composition according to claim 12, wherein the pharmaceutical composition releases the lercanidipine in vitro at a rate of more than about 80% within the first three hours following entry of the pharmaceutical composition into an use environment.

14. The pharmaceutical composition according to claim 12, wherein the pharmaceutical composition releases the lercanidipine in vitro at a rate of more than about 80% within the first hour following entry of the form into an use environment.

15. The pharmaceutical composition according to claim 12, wherein the outer most layer contains at least one material selected from the group consisting of an anionic acrylic co-polymer comprises methacrylic acid and methylmethacrylate monomers, ethyl cellulose, and Aquacoat.

16. The pharmaceutical composition according to claim 12, wherein the outer most layer is at least 5% of the weight of the core.

17. The pharmaceutical composition of claim 12, wherein the outer most layer comprises copolymers of acrylic and methacrylic esters with high and low permeability and combinations thereof.

18. The pharmaceutical composition of claim 12, wherein the outer most layer comprises a combination of copolymers of acrylic and methacrylic esters with high and low permeability.

19. The pharmaceutical composition of claim 20, wherein the weight ratio of the copolymers of acrylic and methacrylic esters with high permeability to the copolymers of acrylic and methacrylic esters with low permeability is from about 70:30 and about 100:0.

20. The pharmaceutical composition of claim 12, wherein the outer most layer comprises copolymers of acrylic and methacrylic esters with high permeability.

21. The pharmaceutical composition of claim 12, wherein the outer layer comprises an release modifying acrylic polymer selected from the group consisting of Eudragit R® RL 30 D and Eudragit R® RS 30 D and combinations thereof.

22. The pharmaceutical composition of claim 12, wherein the outer layer comprises a combination of Eudragit R® RL 30 D and Eudragit R® RS 30 D.

23. The pharmaceutical composition of claim 12, wherein the weight ratio of the Eudragit R® RL 30 D to the Eudragit R® RS 30 D is from about 70:30 and about 100:0.

24. The pharmaceutical composition of claim 12, wherein the outer layer comprises Eudragit R® RL 30 D.

25. The pharmaceutical composition of claim 12, wherein the outer layer further comprises a hydroxypropylmethyl-cellulose film coating.

26. The pharmaceutical composition of claim 12, wherein lercanidipine is present in an amount from about 0.001 to about 0.2 mg per mg of the total composition.

27. A modified release lercanidipine composition comprising:
(1) an immediate release core comprising:
(a) an inert core,
(b) a first layer substantially enveloping the inert core, wherein the first layer comprises comprising (i) lercanidipine, (ii) a surfactant, (iii) a binder, and
(c) optionally a second layer comprising a film coating; and
(2) an outer-most layer comprising at least one release modifying acrylic polymer, wherein the modified release lercanidipine composition, wherein upon administration of the composition to a patient the composition provides for sustained release of lercanidipine, such that the in vivo plasma concentration of lercanidipine is from about 0.1 ng/ml to about 0.4 ng/ml for a period of about 20 to about 25 hours following administration.

28. An oral dosage form comprising:
(i) a plurality of immediate release lercanidipine beads, and
(ii) a plurality of modified release lercanidipine beads, wherein the ratio of (i) to (ii) on a mass basis is from about 1:1 to about 1:20.

29. The oral dosage form of claim 28, wherein the oral dosage form is suitable for once daily oral administration.

30. The oral dosage form of claim 28, wherein the total dosage of lercanidipine is from about 1 to about 80 mg per dose.

31. The oral dosage form of claim 28, wherein the amount of lercanidipine present in the immediate release lercanidipine beads is from about 1 to about 10 mg and the amount of lercanidipine present in the modified release lercanidipine beads is from about 1 to about 80 mg.

32. The oral dosage form of claim 28, wherein upon administration of the dosage form to a patient, the immediate release beads are released at the pH of the stomach and provide for a rapid increase in the plasma concentration of lercanidipine, and wherein the modified release beads are released to provide for sustained release of the lercanidipine at therapeutic plasma concentrations.

33. The oral dosage form of claim 28, wherein the release of the immediate release beads results in a maximum in vivo plasma concentration of lercanidipine from about 8 to about 12 ng/ml, within a period of about 1 to about 3 hours following administration of the dosage form to a patient.

34. The oral dosage form of claim 28, wherein the release of the modified release beads results in an in vivo plasma concentration of lercanidipine greater than about 0.1 ng/ml to about 0.4 ng/ml for a period from about 20 to about 24 hours following administration of the dosage form to a patient.
